(19) United States
(12) Patent Application Publication
(10) Pub. No.: US 2007/0282392 A1
(43) Pub. Date: Dec. 6, 2007

(54) METHOD AND SYSTEM FOR PROVIDING HEARING ASSISTANCE TO A USER

(75) Inventors: Francois Callias, Fontaines (CH); Evert Dijkstra, Fontaines (CH)

Correspondence Address: ROBERTS, MLOTKOWSKI & HOBBES, P. O. BOX 10064, MCLEAN, VA, 22102-8064, US

(73) Assignee: Phonak AG, Staefa (CH)

(21) Appl. No.: 11/420,828

(22) Filed: May 30, 2006

(51) Int. Cl. A61N 1/00

(52) U.S. Cl. 607/55

(57) ABSTRACT

There is provided a method for providing hearing assistance to a user, comprising: capturing audio signals by a microphone arrangement (18) and transmitting the audio signals by a transmission unit (10) via a modulated wireless audio link (30) to a receiver unit (12) and receiving the audio signals at the receiver unit (12); analyzing the amplitude of the received audio signals by an analyzer unit (34) of the receiver unit (12); dynamically adjusting by a gain control unit (36) located in the receiver unit (12) the gain applied to the received audio signals according to the result of the analysis by the analyzer unit (34), wherein the gain is equal to or larger than a first value (G_{H}) if the amplitude of the received audio signals is equal to or larger than a first threshold (U_{1H}) and is reduced to a finite value less than said first value if the amplitude of the received audio signals is less than said first threshold (U_{1H}); and stimulating the user's hearing by stimulating means (14, 16) worn at or in the user's ear according to the audio signals amplified according to the gain set by the gain control unit (36).

### BACKGROUND OF THE INVENTION

1. Field of the Invention

The present invention relates to a method for providing hearing assistance to a user; it also relates to a corresponding system. In particular, the invention relates to a system comprising a microphone arrangement for capturing audio signals; a transmission unit for transmitting the audio signals via a wireless audio link to a receiver unit for receiving the audio signals; a gain control unit located in the receiver unit for applying a gain to the received audio signals; and means to be worn at or in the user's ear for stimulating the user's hearing according to the audio signals amplified according to the gain set by the gain control unit.

2. Description of Related Art

Usually in such systems the wireless audio link is an FM radio link. The benefit of such systems is that sound captured by a remote microphone at the transmission unit can be presented at a high sound pressure level to the hearing of the user wearing the receiver unit at his ear(s).

According to one typical application of such wireless audio systems, the stimulating means is loudspeaker which is part of the receiver unit or is connected thereto. Such systems are particularly helpful for being used in teaching normal-hearing children suffering from auditory processing disorders (APD), wherein the teacher's voice is captured by the microphone of the transmission unit, and the corresponding audio signals are transmitted to and are reproduced by the receiver unit worn by the child, so that the teacher's voice can be heard by the child at an enhanced level, in particular with respect to the background noise level prevailing in the classroom. It is well known that presentation of the teacher's voice at such enhanced level supports the child in listening to the teacher.

Usually in such systems the audio signals received by the receiver are amplified at a given constant gain for being reproduced by the output transducer. Such receiver unit has as a drawback that due to the constant gain the audio signals received from the remote microphone are amplified irrespective of whether they are desired by the user (e.g. if the teacher is silent there is no benefit to the user by receiving audio signals from the remote microphone, which then may consist primarily of noise).

According to another typical application of wireless audio systems the receiver unit is connected to or integrated into a hearing instrument, such as a hearing aid. The benefit of such systems is that the microphone of the hearing instrument can be supplemented or replaced by the remote microphone which produces audio signals which are transmitted wirelessly to the FM receiver and thus to the hearing instrument. In particular, FM systems have been standard equipment for children with hearing loss in educational settings for many years. Their merit lies in the fact that a microphone placed a few inches from the mouth of a person speaking receives speech at a much higher level than one placed several feet away. This increase in speech level corresponds to an increase in signal-to-noise ratio (SNR) due to the direct wireless connection to the listener's amplification system. The resulting improvements of signal level and SNR in the listener's ear are recognized as the primary benefits of FM radio systems, as hearing-impaired individuals are at a significant disadvantage when processing signals with a poor acoustical SNR.

Most FM systems in use today provide two or three different operating modes. The choices are to get the sound from: (1) the hearing instrument microphone alone, (2) the FM microphone alone, or (3) a combination of FM and hearing instrument microphones together.

Usually, most of the time the FM system is used in mode (3), i.e. the FM plus hearing instrument combination (often labeled “FM+M” or “FM+ENV” mode). This operating mode allows the listener to perceive the speaker's voice from the remote microphone with a good SNR while the integrated hearing instrument microphone allows to listener to also hear environmental sounds. This allows the user/listener to hear and monitor his own voice, as well as voices of other people or environmental noise, as long as the loudness balance between the FM signal and the signal coming from the hearing instrument microphone is properly adjusted. The so-called “FM advantages” measures the relative loudness of signals when both the FM signal and the hearing instrument microphone are active at the same time. As defined by the ASHA (American Speech-Language-Hearing Association 2002), FM advantage compares the levels of the FM signal and the local microphone signal when the speaker and the user of an FM system are spaced by a distance of two meters. In this example, the voice of the speaker will travel 30 cm to the input of the FM microphone at a level of approximately 80 dB-SPL, whereas only about 65 dB-SPL will remain of this original signal after traveling the 2 m distance to the microphone in the hearing instrument. The ASHA guidelines recommend that the FM signal should have a level 10 dB higher than the level of the hearing instrument's microphone signal at the output of the user's hearing instrument.

When following the ASHA guidelines (or any similar recommendation), the relative gain, i.e. the ratio of the gain applied to the audio signals produced by the FM microphone and the gain applied to the audio signals produced by the hearing instrument microphone, has to be set to a fixed value in order to achieve e.g. the recommended FM advantage of 10 dB under the above-mentioned specific conditions. Accordingly, heretofore—depending on the type of hearing instrument used—the audio output of the FM receiver has been adjusted in such a way that the desired FM advantage is either fixed or programmable by a professional, so that during use of the system the FM advantage—and hence the gain ratio—is constant in the FM+M mode of the FM receiver.

EP 0 563 194 B1 relates to a hearing system comprising a remote microphone/transmitter unit, a receiver unit worn at the user's body and a hearing aid. There is radio link between the remote unit and the receiver unit, and there is an inductive link between the receiver unit and the hearing aid. The remote unit and the receiver unit each comprise a microphone, with the audio signals of theses two microphones being mixed in a mixer. A variable threshold noise-gate or voice-operated circuit may be interposed between the microphone of the receiver unit and the mixer, which circuit is primarily to be used if the remote unit is in a line-input mode, i.e. the microphone of the receiver then is not used.

WO 97/21325 A1 relates to a hearing system comprising a remote unit with a microphone and an FM transmitter and an FM receiver connected to a hearing aid equipped with a microphone. The hearing aid can be operated in three modes, i.e. “hearing aid only”, “FM only” or “FM+M”. In the FM+M mode the maximum loudness of the hearing aid microphone audio signal is reduced by a fixed value between 1 and 10 dB below the maximum loudness of the FM microphone audio signal, for example by 4 dB. Both the FM microphone and the hearing aid microphone may be provided with an automatic gain control (AGC) unit.

WO 2004/100607 A1 relates to a hearing system comprising a remote microphone, an FM transmitter and left- and right-ear hearing aids, each connected with an FM receiver. Each hearing aid is equipped with a microphone, with the audio signals from remote microphone and the respective hearing aid microphone being mixed in the hearing aid. One of the hearing aids may be provided with a digital signal processor which is capable of analyzing and detecting the presence of speech and noise in the input audio signal from the FM receiver and which activates a controlled inverter if the detected noise level exceeds a predetermined limit when compared to the detected level, so that in one of the two hearing aids the audio signal from the remote microphone is phase-inverted in order to improve the SNR.

WO 02/30153 A1 relates to a hearing system comprising an FM receiver connected to a digital hearing aid, with the FM receiver comprising a digital output interface in order to increase the flexibility in signal treatment compared to the usual audio input parallel to the hearing aid microphone, whereby the signal level can easily be individually adjusted to fit the microphone input and, if needed, different frequency characteristics can be applied. However, is not mentioned how such input adjustment can be done.

Contemporary digital hearing aids are capable of permanently performing a classification of the present auditory scene captured by the hearing aid microphones in order to select the hearing aid operation mode which is most appropriate for the determined present auditory scene. Examples for such hearing aids with auditory scene analyses can be found in US 2002/0037087, US 2002/0090098, CA 2 439 427 A1 and US 2002/0150264.

EP 0 483 701 A2 (corresponding to AU 8 586 691 A) relates to a hearing aid wherein the gain applied to the hearing aid microphone signal is controlled in such a manner that if the level of the microphone signal is below a first threshold the gain is kept constant at a first value, if the level of the microphone signal is higher than the first threshold but lower than a second threshold, the gain increases with increasing microphone signal level, and if the level of the microphone signal is above the second threshold, the gain is kept constant at a second value higher than the first value. By varying the gain applied to the microphone signal as a function of the microphone signal amplitude in such a manner, noise occurring at low microphone signal levels can be reduced or eliminated. Such gain control is also labeled “soft squelch”.

U.S. Pat. No. 3,928,733 relates to a hearing aid, wherein the gain applied to the microphone signals is controlled in such a manner that if the microphone signal level is below a first threshold the microphone is muted, if the microphone signal level is between the first threshold and a second threshold, the gain increases with increasing microphone signal level, and if the microphone signal level is above the second threshold, the gain is kept constant. Also in this case the gain control serves to eliminate noise at low microphone signal levels.

FM radio communication systems, such as walkie-talkies, usually are equipped with a squelch function by which the received signal in the receiver is muted, if the level of the received demodulated signal is too low in order to avoid perception of excessive noise by the user of the receiver.

Usually also FM (or inductive) receivers used for hearing instruments are equipped with such a squelch function in order to mute the receiver audio signal if the distance between the transmitter and the receiver is too large, so that perception of excessive noise from the receiver is avoided. However, such devices have as a drawback that the absence of any perceivable sound during times when the squelch function is active is perceived by the user as being unpleasant. In particular, the user may feel that the receiver does not properly work.

U.S. Pat. No. 5,734,976 relates to an examples of an FM receiver for a hearing instrument wherein hard muting of the amplifier of the receiver in case of excess noise caused by large distance is implemented. EP 1 619 926 A1 mentions that a squelch function may be implemented in inductive receivers.

It is an object of the invention to provide for a method for providing hearing assistance to a user using a system comprising a remote microphone arrangement, a transmission unit and a receiver unit, wherein the remote microphone audio signals are transmitted via a modulated wireless audio link to the receiver unit from which the audio signals are provided to the user's hearing via stimulating means worn at the user's ear. It is a further object of the invention to provide for a corresponding hearing assistance system.

### SUMMARY OF THE INVENTION

According to the invention, these objects are achieved by a method as defined in claim 1 and a system as defined in claim 22, respectively.

The invention is beneficial in that, by dynamically reducing the gain applied to the received audio signals to finite values for low values of the amplitude of the received audio signals, a reduction or elimination of the perception of noise resulting from electronic noise due to a relatively large distance between the transmission unit and the receiver unit and/or environmental acoustic noise picked up by the remote microphone at low voice levels can be reduced or eliminated while still even in this regime of reduced gain a perceivable sound signal is provided to the user, whereby feelings of discomfort due to absence of any sound signals from the receiver unit—as it is the case for the usual (hard) squelch function—can be avoided. In other words, by automatically decreasing the output signal amplitude at low levels of the received audio signals to low but still finite values an efficient and user-comfortable noise reduction system is provided.

According to a preferred embodiment, the gain is kept constant at a high value if the amplitude of the received audio signals is equal to or larger than the first threshold, the gain increases with increasing amplitude of the received audio signals if the amplitude of the received audio signals is between a second threshold lower than the first threshold and the first threshold, and the gain is kept constant at a low value if the amplitude of the received audio signals is lower than the second threshold. Preferably, between the first and second threshold the gain increases at an expansion factor of 2:1 with the amplitude level of the received audio signals, i.e. the output level increases by 2 dB each time the level of the amplitude of the received audio signals increases by 1 dB.

The gain may be varied within a dynamic range of 20 dB or less, preferably 12 dB or less, as a monotonous function of the amplitude of the received audio signals.

Preferably, for decreasing amplitude of the received audio signals the gain is reduced with a relatively long time constant between 50 and 300 msec, for example 100 msec, in order to achieve smooth transitions between different gain levels for avoiding distortion of the amplified audio signals. On the other hand, for increasing amplitude of the received audio signals the gain preferably is increased with a relatively short time constant between 1 and 10 msec, typically a few msec, in order to avoid some loss of the voice during times when the speaker begins to speak.

These and further objects, features and advantages of the present invention will become apparent from the following description when taken in connection with the accompanying drawings which, for purposes of illustration only, show several embodiments in accordance with the present invention.

### BRIEF DESCRIPTION OF THE DRAWINGS

FIG. 1 is a block diagram of an example of a wireless hearing assistance system according to the invention;

FIG. 2 is a block diagram of one example of the electronic module of the receiver unit;

FIG. 3 is a block diagram of an alternative example of the electronic module of the receiver unit;

FIG. 4 is an example of the expansion characteristic provided by the electronic module of the receiver unit, with the output amplitude being shown as a function of the received audio signal level; and

FIG. 5 shows an example of the gain applied by the electronic module of the receiver unit as a function of the amplitude of the received audio signal level.

### DETAILED DESCRIPTION OF THE INVENTION

FIG. 1 is a block diagram of a hearing assistance system comprising a transmission unit 107 a receiver unit 12 and an output transducer 14, 16 for stimulating a user's hearing.

The transmission unit 10 comprises a microphone arrangement 18, which preferably consists of two spaced-apart microphones for achieving acoustic beam forming, a transmitter module 20 and an antenna 22. The receiver unit 12 comprises an antenna 24 and an electronic module 26. The output of the receiver unit 12 may be directly connected to an output transducer 14. As a modification, the output transducer 14 may be part of receiver unit 12. According to an alternative embodiment, the receiver unit 12 may be connected—usually via an audio shoe—mechanically and electronically to a hearing instrument 28 comprising an output transducer 16. In this case the output of the receiver unit 12 will be connected to an audio input of the hearing instrument 28. According to an alternative embodiment, the elements of the receiver unit 12 may be integrated within the hearing instrument 28.

In any case, the output transducer 14, 16, which usually will be of the electro-acoustic type, i.e. a loudspeaker, will be worn at the user's ear. To this end, it could be located behind the ear (BTE), in the ear (IPE) or completely in the ear (CIC). Also the receiver unit 12 will be worn at or close to the user's ear.

The transmission unit 10 and the receiver unit 12 are adapted to establish a wireless audio link 30, usually an FM (frequency modulation) radio link between the transmission unit 10 and the receiver unit 12 for transmitting audio signals captured by the microphone 18 from the transmission unit 10 to the receiver unit 12. The signals received by the antenna 24 of the receiver unit 12 undergo signal processing in the electronic module 26 and finally are provided as audio signals to the output transducer 14, 16 for stimulating the user's hearing according to the audio signals received by the receiver unit 12.

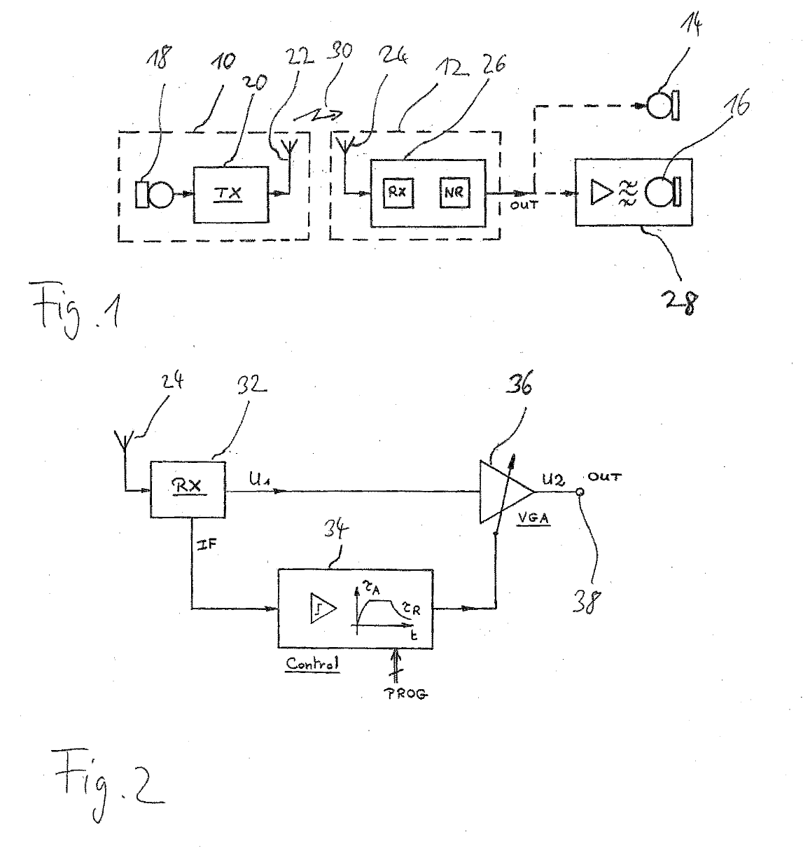

An example of the electronic module 26 of FIG. 1 is shown in FIG. 2. The electronic module 26 comprises a demodulator 32, an analyzer unit 34 and a variable gain amplifier 36. The demodulator 32 has two outputs, one carrying the demodulated audio signal U1 received from the transmission unit 10, and the other one carrying an IF signal which is a down-conversion of the received radio signal to a lower intermediate frequency. The demodulated audio signal U1 is supplied to the variable gain amplifier 36 where it is amplified and delivered as an audio output signal U2 at the output 38 of the receiver unit 12 for being supplied to the output transducer 14 or to the audio input of the hearing instrument 28. The IF signal is supplied to the analyzer unit 34 by which the modulation width of the IF signal is measured. The modulation width of the IF signal is proportional to the amplitude of the demodulated audio signal U₁. The analyzer unit 34 serves to set the gain applied by the amplifier 36 according to the measured modulation width of the IF signal—and thus according to the amplitude of the demodulated audio signal. To this end, the analyzer unit 34 provides an output signal as a function of the measured modulation width of the IF signal in order to control the variable gain amplifier 36 accordingly.

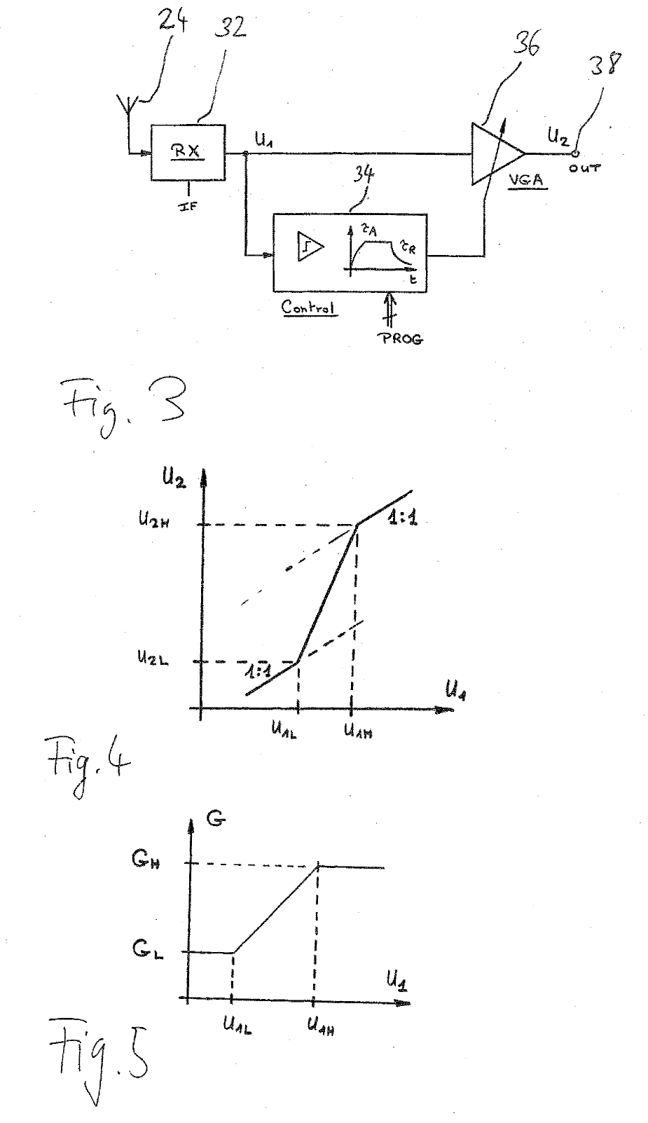

FIG. 3 shows an alternative embodiment of the electronic module 26 wherein the analyzer unit 34 is provided with the demodulated audio signal U₁ rather than with the IF signal in order to directly analyze the amplitude of the demodulated audio signal U₁ for controlling the amplifier 36 accordingly.

FIG. 5 shows an example of the gain applied by the amplifier 36 as a function of the amplitude of the demodulated audio signal U₁ under the control of the analyzer unit 34. FIG. 4 shows the corresponding amplitude of the audio output signal U₂ as a function of the amplitude of the demodulated audio signal U₁. According to FIGS. 4 and 5 the gain is constant at a high value G_{H}=20 Log(2/U₁) if the amplitude of the demodulated audio signal U₁ is equal to larger than a threshold U_{1H}, while the gain is constant at a low value G_{L}=20 Log(U₂/U₁) if the amplitude of the demodulated audio signal U₁ is equal to or less than a lower threshold U_{1L}. If the amplitude of the demodulated audio signal U₁ is between the low threshold and the high threshold, the gain increases with increasing amplitude of the demodulated audio signal U₁, preferably at an expansion factor of 2:1, i.e. the output level U₂ increases by 2 dB each time the input level U₁ increases by 1 dB.

As an example, the dynamic range within which the gain is varied could be 12 dB. Preferably, the range of the gain variation is less than 20 dB in order to ensure that even for the lowest gain the user will be able to perceive a sound signal so that he does not get the feeling that the system does not work properly.

The time constants of the analyzer unit are selected such that for decreasing amplitude of the audio signal U₁ the gain applied by the amplifier 36 is reduced with a relatively long time constant of, for example, 100 msec, while in the case in which the amplitude of the audio signal U₁ is increasing, the gain applied by the amplifier 36 is increased quickly at a time constant of a few msec. Thereby it is ensured that for falling audio signal amplitude smooth transitions between different gains are made in order to avoid distortions while for rising audio signal amplitudes the gain is quickly increased to the necessary higher value so that the voice of the speaker using the microphone arrangement 18 will be captured by the output transducer 14, 16 almost immediately when the speaker starts speaking.

According to the invention, the gain applied to the audio signals received in the receiver unit 12 is varied dynamically according to the amplitude of the received audio signals measured by the analyzer unit in such a manner that for low input audio signal levels the audio signal output level is reduced with respect to the output level for high levels of the audio input signal, while maintaining the audibility of the demodulated signals even at relatively low audio input signals. Thereby both electronic noise present in the system at least at relatively large distances between the transmission unit 10 and the receiver unit 12 and surrounding acoustic noise picked up by the microphone 18 of the transmission unit can be reduced or eliminated while still an audible signal is presented to the user so that he does not have an uncomfortable feeling that the system sometimes does not work properly.

While various embodiments in accordance with the present invention have been shown and described, it is understood that the invention is not limited thereto, and is susceptible to numerous changes and modifications as known to those skilled in the art. Therefore, this invention is not limited to the details shown and described herein, and includes all such changes and modifications as encompassed by the scope of the appended claims.

What is claimed is:

1. A method for providing hearing assistance to a user, comprising:
(a) capturing audio signals by a microphone arrangement and transmitting said audio signals by a transmission unit via a modulated wireless audio link to a receiver unit and receiving said audio signals at said receiver unit;
(b) analyzing an amplitude of said received audio signals by an analyzer unit of said receiver unit;
(c) dynamically adjusting by a gain control unit located in said receiver unit a gain applied to said received audio signals according to a result of an analysis by said analyzer unit, wherein said gain is equal to or larger than a first value if said amplitude of said received audio signals is equal to or larger than a first threshold and is reduced to a finite value less than said first value if said amplitude of said received audio signals is less than said first threshold; and
(d) stimulating a hearing of said user by stimulating means worn at or in an ear of said user according to said audio signals amplified according to said gain set by said gain control unit.

2. The method of claim 1, wherein said gain is constant at said first value if said amplitude of said received audio signals is equal to or larger than said first threshold.

3. The method of claim 2, wherein said gain is constant at a second value lower than said first value, if said amplitude of said received audio signals is less than a second threshold which is lower than said first threshold.

4. The method of claim 3, wherein said gain increases with increasing amplitude of said received audio signals from said second value to said first value, if said amplitude of said received audio signals is between said first threshold and said second threshold.

5. The method of claim 4, wherein said gain increases at an expansion factor of 2:1 with an amplitude level of said received audio signals, if said amplitude of said received audio signals is between said first threshold and said second threshold.

6. The method of claim 1, wherein said gain is varied by said gain control unit within a dynamic range of 20 dB or less as a monotonous function of said amplitude of said received audio signals.

7. The method of claim 6, wherein said gain is varied by said gain control unit within a dynamic range of 12 dB or less as a monotonous function of said amplitude of said received audio signals.

8. The method of claim 1, wherein for decreasing amplitude of said received audio signals said gain is reduced by said gain control unit with a time constant between 50 and 300 msec.

9. The method of claim 1, wherein for increasing amplitude of said received audio signals said gain is increased by said gain control unit with a time constant between 1 and 10 msec.

10. The method of claim 1, wherein said analyzer unit analyses said amplitude of said received audio signals by measuring an amplitude of said audio signals after having been demodulated.

11. The method of claim 1, wherein said analyzer unit analyses said amplitude of said received audio signals by measuring an modulation width of an intermediate frequency signal having a frequency lower than a carrier of said received audio signals.

12. The method of claim 1, wherein said gain control unit is a variable gain amplifier.

13. The method of claim 1, wherein said stimulating means is part of a hearing instrument to which said receiver unit is mechanically and electrically connected.

14. The method of claim 1, wherein said stimulating means is part of a hearing instrument into which said receiver unit is integrated.

15. The method of claim 1, wherein said stimulating means is part of said receiver unit.

16. The method of claim 1, wherein said stimulating means is connected to said receiver unit.

17. The method of claim 1, wherein said analyzer unit outputs a gain control signal according to said result of said analysis and wherein said gain control signal is applied to said gain control unit.

18. The method of claim 1, wherein said microphone arrangement is part of said transmission unit.

19. The method of claim 1, wherein said stimulating means is a loudspeaker.

20. The method of claim 1, wherein said modulated wireless audio link is a Radio Frequency (RF) link.

21. The method of claim 20, wherein said Radio Frequency link is a Frequency Modulation (FM) link

22. A system for providing hearing assistance to a user, comprising a microphone arrangement for capturing audio signals; a transmission unit for transmitting said audio signals via a modulated wireless audio link to a receiver unit for receiving said audio signals; an analyzer unit located in said receiver unit for analyzing an amplitude of said received audio signals; a gain control unit located in said receiver unit for dynamically adjusting a gain applied to said audio signals according to a result of an analysis by the analyzer unit; wherein said gain control unit is designed such that said gain is equal to or larger than a first value if said amplitude of said received audio signals is equal to or larger than a first threshold and is reduced to a finite value less than said first value if said amplitude of said received audio signals is less than said first threshold; and means to be worn at or in an ear of said user for stimulating a hearing of said user according to said audio signals amplified according to said gain set by said gain control unit.
